(19) United States
(12) Patent Application Publication
(10) Pub. No.: US 2008/0189837 A1
(43) Pub. Date: Aug. 14, 2008

(54) Swimming goggles

(76) Inventor: Herman Chiang, Chung-Ho City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/882,967

(22) Filed: Aug. 8, 2007

Related U.S. Application Data
(63) Continuation-in-part of application No. 11/705,015, filed on Feb. 12, 2007.

(30) Foreign Application Priority Data
Jun. 13, 2007 (TW) 096209739

(51) Int. Cl. A61F 9/02

(52) U.S. Cl. 2/431; 2/426

(57) ABSTRACT

Swimming goggles include a soft left lens frame, a soft right lens frame, a soft connecting portion between the left lens frame and the right lens frame, rigid eyeglasses, rigid strap positioning frames, and fasteners. Each strap positioning frame includes a housing, two pegs, and two branches extending reverse to the pegs. Each peg has at least a holding portion. The left lens frame and the right lens frame respectively define at least an embedding groove for engaging with the holding portion, whereby the left lens frame and the right lens frame firmly wraps the strap positioning frames. The fasteners include adjusting buckles with a head strap extending therethrough. Length of the head strap is adjustable by pressing the adjusting buckles.

### RELATED APPLICATIONS

This application is continuation-In-Part Application of U.S. patent application Ser. No. 11/705,015.

### BACKGROUND OF THE INVENTION

1. Field of the Invention

The present invention relates to swimming goggles, and in particular to swimming goggles which prevent water leakage and facilitates adjustment of a head strap thereof.

2. Related Art

As disclosed in U.S. patent Ser. No. 11/705,015, swimming goggles are generally manufactured as following steps: eyeglasses in left and right lens frames are preliminarily positioned by rigid strap positioning frames; and soft material unitarily wraps rims of the eyeglasses and parts of the strap positioning frames. In this way, when a head strap assembled on the strap positioning frames is worn, the strap positioning frames stands up force exerted thereon without influence to the left and right lens frames, avoiding water leakage owing to deformation of the left and right lens frames. Moreover, the soft left and right lens frames provide comfortable wearing and touching.

The instant invention is directed to improve the design above, and have advantages of reliable connection of rigid strap positioning frame and soft lens frames and enhanced of water leakage and adjustment of the head strap.

### SUMMARY OF THE INVENTION

Accordingly, an object of the present invention is to provide swimming goggles which have lens frames not subject to external force and are designed ergonomically to be worn comfortably, and which prevent water leakage and facilitate to adjust a head strap thereof.

Another object of the present invention is to provide swimming goggles which allows to adjust a head strap ergonomically.

The swimming goggles of the present invention comprise a soft left lens frame, a soft right lens frame, a soft connecting portion between the left lens frame and the right lens frame, rigid eyeglasses, rigid strap positioning frames, and fasteners. The fasteners include adjusting buckles with a head strap extending therethrough. The head strap defines stopping grooves therein. The eyeglasses and the strap positioning frames are preliminarily assembled together. The left lens frame and the right lens frame are unitarily formed to connect with each other and envelop rims of the eyeglasses and parts of the strap positioning frames. The eyeglasses form positioning portions. Each strap positioning frame includes a housing, at least a peg, and at least a branch extending reverse to the pegs. The pegs form latches thereon for engaging with the positioning portions of the eyeglasses. Each peg has at least a holding portion. The left lens frame and the right lens frame respectively define at least an embedding groove for engaging with the holding portion, whereby the left lens frame and the right lens frame firmly wraps the strap positioning frames in molding.

Each adjusting buckle includes a pressing element pivotable to the branches in a leverage fashion, a resilient element for providing return force for the pressing element, and a buckle cover assembling on the branches. The pressing element forms an assembling axis for assembling on the axis holes. The pressing element has an abutting edge on an end thereof for stopping the stopping grooves of the head strap, and a button on another end thereof. The buckle cover defines a buckle hole for corresponding to the button.

### BRIEF DESCRIPTION OF THE DRAWINGS

FIG. 1 is an exploded view of swimming goggles of the present invention.

FIGS. 2A-2C show the swimming goggles of FIG. 1 being assembled step by step.

FIG. 3 is an assembled view of the swimming goggles of FIG. 1.

FIG. 4 is a front view of the swimming goggles of FIG. 3.

FIG. 5 is a cross-sectional view taken along the line 5-5 in FIG. 4.

FIG. 6 is a partially enlarged view of FIG. 5.

### DESCRIPTION OF THE PREFERRED EMBODIMENTS

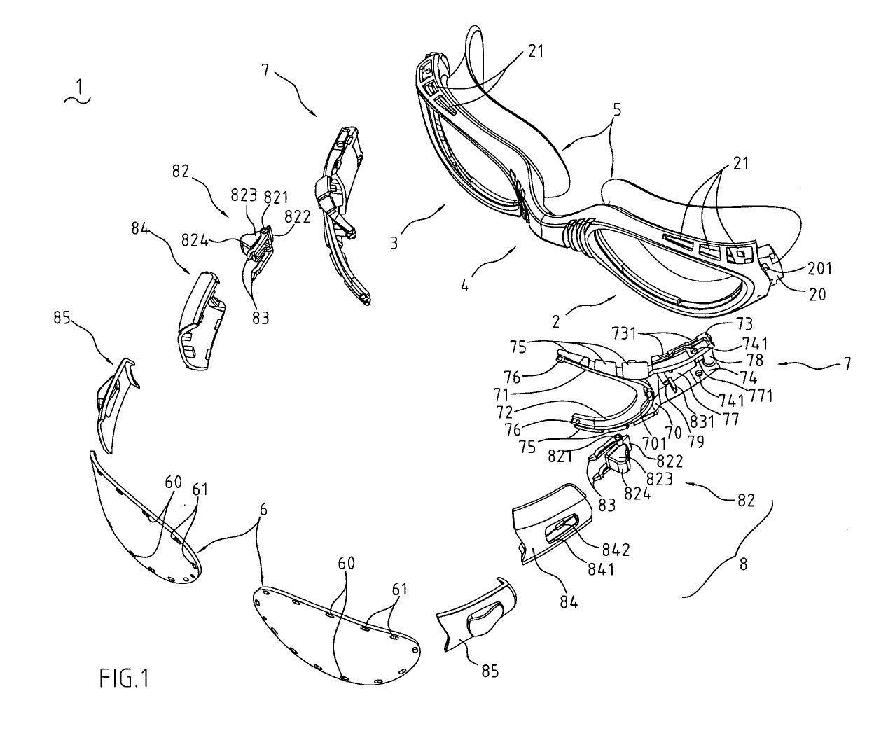

Referring to FIG. 1, swimming goggles 1 in accordance with the present invention comprise a left lens frame 2, a right lens frame 3, a connecting portion 4, pads 5, eyeglasses 6, strap positioning frames 7 and fasteners 8. The left lens frame 2, the right lens frame 3, the connecting portion 4 and the pads 5 are made of soft material. The eyeglasses 6 and the strap positioning frames 7 are made of rigid material. In one embodiment, the connecting portion 4, the left lens frame 2 and the right lens frame 3 are made of the same soft material and are integrally formed. The left lens frame 2 and the right lens frame 3 are respectively formed integrally with the pads 5. In one embodiment, the soft material is silica gel, and the rigid material is PP. The left lens frame 2 and the right lens frame 3 are assembled together by unitarily forming. During one time molding of the left lens frame 2, the right lens frame 3, the connecting portion 4, the pads 5, the eyeglasses 6, the strap positioning frames 7 and the fasteners, rims of the eyeglasses 6 are wrapped and the strap positioning frames 7 are partially exposed out (this will be described in detail). The left lens frame 2 and the right lens frame 3 respectively form adjoining bases 20 at outward sides thereof. Each adjoining base 20 defines an assembling groove 201 for assembling the strap positioning frames 7. A plurality of embedding grooves 21 is defined in the left lens frame 2 and the right lens frame 3 for holding the strap positioning frames 7.

Each eyeglass 6 has positioning portions 60 on an upper and a lower edges. In this embodiment, the positioning portions 60 are through holes. Injecting holes 61 are formed along edges of the eyeglass 6 and beside the positioning portions 60 for plastic passing therethrough when molding. Each strap positioning frame 7 comprises a housing 70, two pegs 71, 72, two branches 73, 74, and a plurality of holding portions 75. The two pegs 71, 72 unitarily extend from the housing 70, and together form an arc which is a half of the left lens frame 2 or the right lens frame 3 for fitting for edges of the eyeglasses 6. The pegs 71, 72 respectively unitarily form latches 76 on ends thereof for cooperating with the positioning portions 60 to assemble the eyeglasses 6 from inward to outward. In this embodiment, the latches 76 are protuberances.

The branches 73, 74 unitarily extend from the housing 70 and reverse to the pegs 71, 72, and are substantially inclined toward each other and spaced a distance apart from each other. A positioning cover 77 is formed between the branches 73, 74. A shaft 78 straddles on ends of the branches 73, 74. An assembling member 79 is formed on an end of the positioning cover 77 and near the housing 70 for assembling to the assembling groove 20 of the left lens frame 2 or the right lens frame 3. Support plates 831 respectively extend from the positioning cover 77. A slot 771 is defined adjacent the shaft 78. The branches 73, 74 respectively define axis holes 741 therein adjacent the shaft 78.

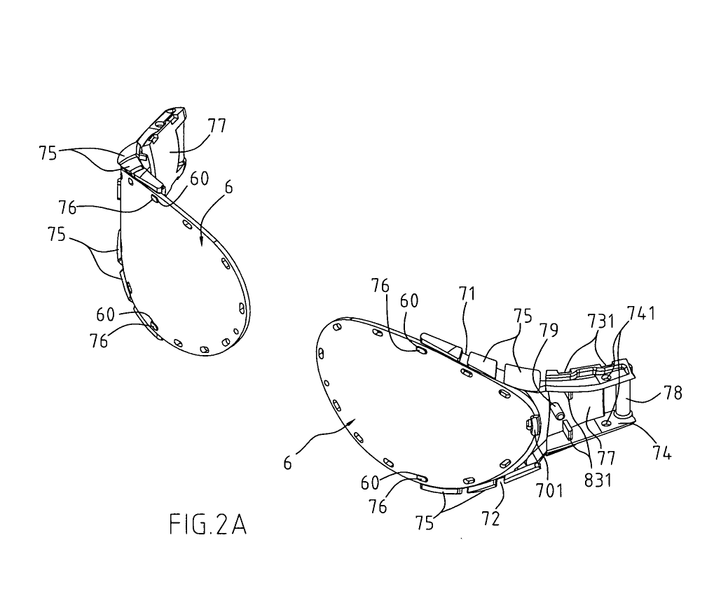

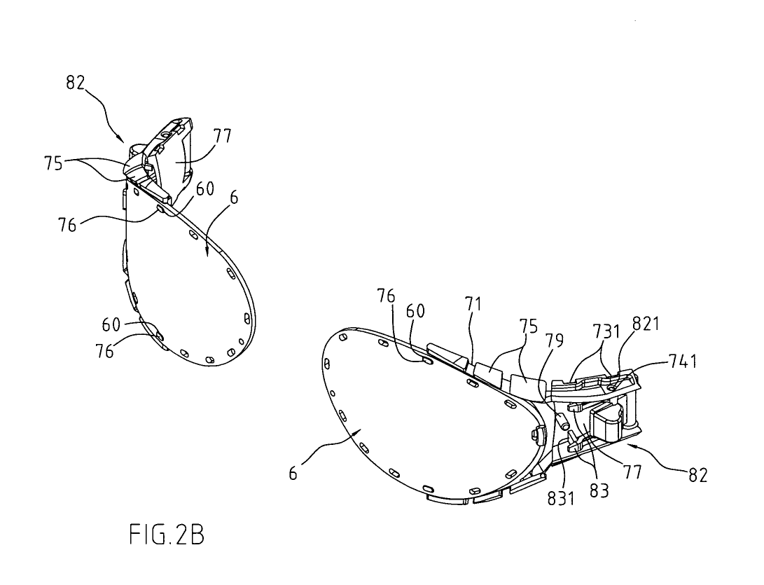

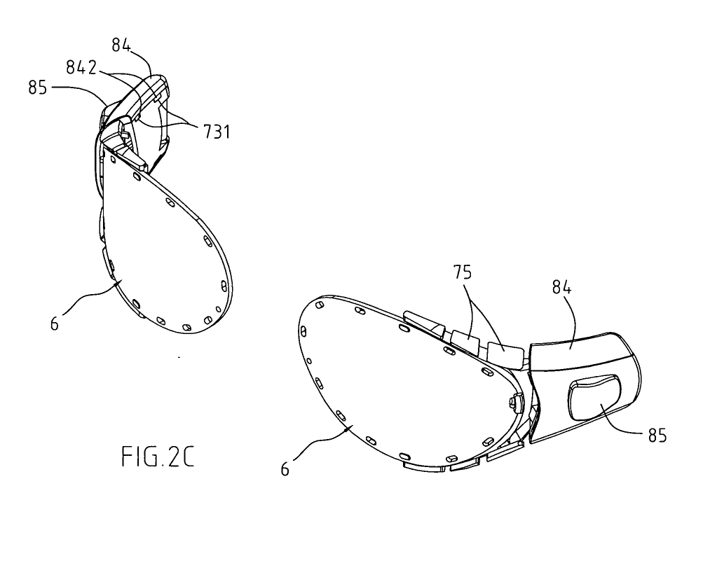

A clip 701 is formed on the housing 70 between the pegs 71, 72 and is L-shaped for biasing against the eyeglasses 6 inward. In other words, during assembly, the eyeglasses 6 and the strap positioning frames 7 are preliminarily positioned, as shown in FIG. 2A. The through holes 60 engage with the protuberances 76 of the branches 73, 74. The eyeglasses 6 are assembled from inward to outward, and the clips 701 bias against the eyeglasses 6 from outward to inward for fixing the eyeglasses 6 preliminarily.

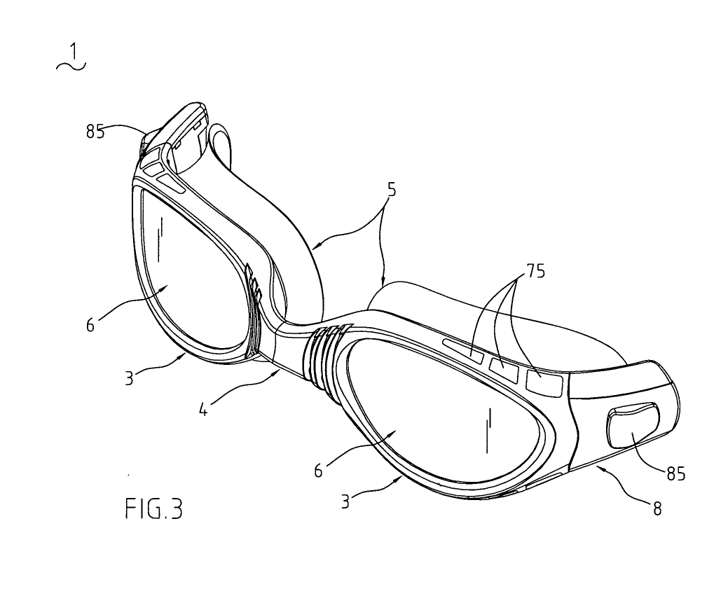

With reference to FIG. 1 to FIG. 6, the fasteners 8 comprise adjusting buckles 81 with a head strap 80 extending therethrough. The head strap 80 defines a plurality of stopping grooves 801 therein.

Each adjusting buckle 81 includes a pressing element 82 pivotable to the branches 73, 74 in a leverage fashion, a resilient element 83, a buckle cover 84 and an outward cover 85. The pressing element 82 forms an assembling axis 821 for assembling on the axis holes 741. The pressing element 82 has an abutting edge 822 on an end thereof for abutting the stopping grooves 801 of the head strap 80, and a button 823 on another end thereof. The buckle cover 84 defines a buckle hole 841 for corresponding to the button 823. The button 823 has a manual portion 824 protruding outward beyond a buckle hole 841 of the buckle cover 84, and cooperate with a protruding portion (not shown) on the outward cover 85 for facilitating manual operation. The outward cover 85 is assembled on outward sides of the buckle cover 84 for covering the buckle cover 84.

The resilient element 83 includes arcuate levers (not labeled) respectively extending from opposite sides of the assembling axis 821. The levers straddle on the support plates 831 (referring to FIG. 2B) such that the button 823 remains a free state in which the button 823 is able to be pressed or return for providing return force for the pressing element 82.

The buckle cover 84 is assembled on the branches 73, 74. The branches 73, 74 respectively define locking holes 731 therein, and the buckle cover 84 forms a locking post 842 for locking with the locking holes 731.

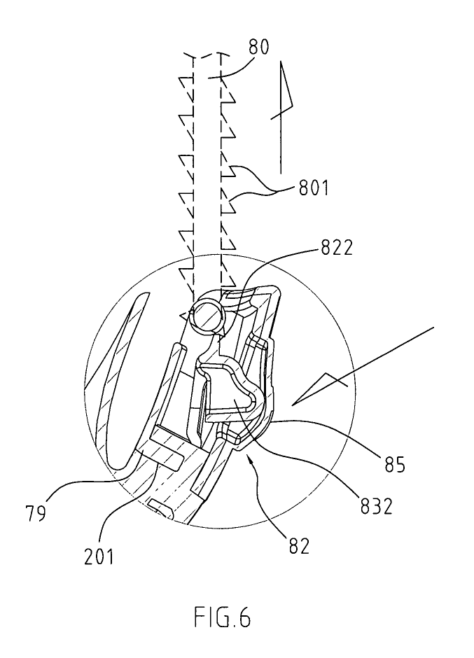

Referring to FIG. 6, when the button 832 is pressed, the abutting edge 822 of the assembling axis 821 moves in a direction reverse to the button 823, and disengages from the stopping grooves 801 of the head strap 80. The head strap 80 moves in a direction of loosening the head strap 80, and the resilient element 83 provides preserved energy. Once the button 823 is freed, the abutting edge 822 of the assembling axis 821 engages to the stopping groove 801 of the head strap 80.

During assembly, the rigid eyeglasses 6 and the pegs 71, 72 are assembled together preliminarily, and are put in a mold. The assembling members 79 are firstly retained to the assembling grooves 201 of the left lens frame 2 and the right lens frame 3. Referring to FIG. 6, the left lens frame 2, the right lens frame 3, the connecting portion 4, and the pads 5 are integrally formed. Plastic is shaped to envelop rims of the eyeglasses 6. The holding portions 75 of the pegs 71, 72 are embedded into the embedding grooves 21 of the left lens frame 2 and the right lens frame 3. The left lens frame 2 and the right lens frame 3 firmly wrap the strap positioning frames 7 when molding due to the cooperation of the holding portions 75 and the embedding grooves 21.

Then the pressing elements 82, the buckle covers 84 and the outward covers 85 are assembled. As shown in FIGS. 2B and 2C in which the left lens frame 2, the right lens frame 3, the connecting portion 4 and the pads 5 are not shown, after the eyeglasses 6 are preliminarily positioned on the pegs 71, 72, the assembling axes 821 of the pressing elements 82 are assembled to the axis holes 741. The resilient elements 83 straddle on the support plates 831 (referring to FIG. 2B). The outward covers 85 and the buckle covers 84 are assembled in sequence. The locking posts 842 of the buckle covers 84 lock with the locking holes 731. Referring to FIG. 2C, protruding portions of the outward covers 85 project outward for easy manual operation.

The eyeglasses 6 are preliminarily positioned on the pegs 71, 72 such that the left lens frame 2 and the right lens frame 3 are not subject to adjustment of the head strap 80. Therefore, the left lens frame 2 and the right lens frame 3 are permitted to be made of soft material for comfortable wearing and touching and effective seepage-proof.

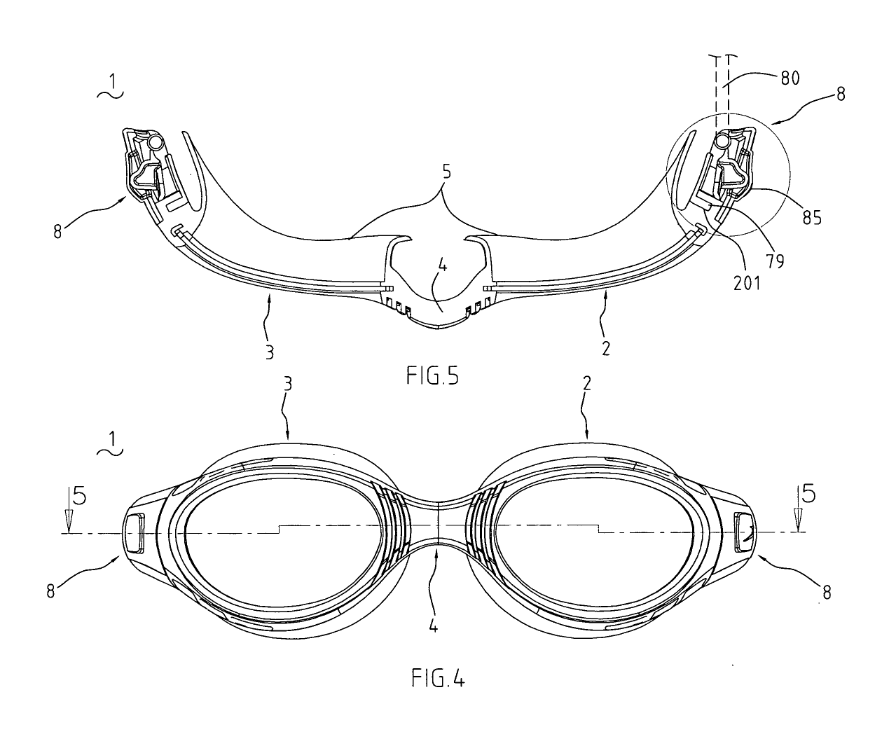

Further referring to FIGS. 4, 5 and 6, in normal state, the abutting edges 822 of the pressing elements 82 abut against the stopping grooves 801 of the head strap 80. The head strap 80 is only allowed to be tightened in normal state. Referring to FIG. 6, in order to loosen the head strap 80, the protruding portions of the outward covers 85 are pressed as arrows in FIG. 6, and bring the buttons 823 to move downward. The abutting edges 822 disengage from the stopping grooves 801 of the head strap 80. In this state, the head strap 80 is allowed to be loosened. Meantime the resilient elements 83 are pressed to reserve energy. When the outward covers 85 are released, the buttons 823 are driven to return by the reserved energy of the resilient elements 83. Length of the head strap 80 can be adjusted in favor of human body.

It is understood that the invention may be embodied in other forms without departing from the spirit thereof. Thus, the present examples and embodiments are to be considered in all respects as illustrative and not restrictive, and the invention is not to be limited to the details given herein.

1. Swimming goggles comprising a soft left lens frame, a soft right lens frame, a soft connecting portion between the left lens frame and the right lens frame, rigid eyeglasses, rigid strap positioning frames, and fasteners having adjusting buckles with a head strap extending therethrough, the head strap defining stopping grooves therein, the eyeglasses and the strap positioning frames being preliminarily assembled together, the left lens frame and the right lens frame being unitarily formed to connect with each other and enveloping rims of the eyeglasses and parts of the strap positioning frames,
wherein the eyeglasses form positioning portions thereon, each strap positioning frame including a housing, at least a peg, and at least a branch extending reverse to the pegs, the pegs forming latches thereon for engaging with the positioning portions of the eyeglasses, each peg having at least a holding portion, the left lens frame and the right lens frame respectively defining at least an embedding groove for engaging with the holding portions whereby the left lens frame and the right lens frame firmly wrap the strap positioning frames in molding.

2. The swimming goggles as claimed in claim 1, wherein the pegs of each strap positioning frame together form an arc which is substantially a half of the left lens frame or the right lens frame for fitting to edges of the eyeglasses.

3. The swimming goggles as claimed in claim 2, wherein the left lens frame and the right lens frame respectively form adjoining bases at outward sides thereof, each adjoining base defining an assembling groove therein for assembling the strap positioning frame.

4. The swimming goggles as claimed in claim 3, wherein the latches of each strap positioning frame include protuberances and are unitarily formed on ends of the pegs, and wherein the positioning portions are through holes and cooperate with the positioning portions to assemble the eyeglasses on the strap positioning frames from inward to outward.

5. The swimming goggles as claimed in claim 4, wherein the branches of each strap positioning frame unitarily extend from the housing and are spaced a distance apart from each other, a positioning cover being formed between the branches, a shaft straddling on ends of the branches, an assembling member being formed on an end of the positioning cover and near the housing for assembling to the assembling groove, a slot being defined adjacent the shaft, the branches respectively defining axis holes therein adjacent the shaft for receiving the adjusting buckles.

6. The swimming goggles as claimed in claim 5, wherein each adjusting buckle includes a pressing element pivotable to the branches in a leverage fashion, a resilient element for providing return force for the pressing element, and a buckle cover assembling on the branches, the pressing element forming an assembling axis for assembling on the axis holes, the pressing element having an abutting edge on an end thereof for stopping the stopping grooves of the head strap, and a button on another end thereof, the buckle cover defining a buckle hole for corresponding to the button.

7. The swimming goggles as claimed in claim 6, wherein the resilient element of each adjusting buckle includes arcuate levers respectively extending from opposite sides of the assembling axis, and wherein support plates respectively extend from the positioning cover, the levers straddling on the support plates, when assembled, the button remaining a free state in which the button is able to be pressed or return.

8. The swimming goggles as claimed in claim 6, wherein the branches of each strap positioning frame respectively define locking holes therein, and the buckle cover of each adjusting buckle forms locking posts for locking with the locking holes.

9. The swimming goggles as claimed in claim 6, wherein each adjusting buckle further comprises an outward cover assembled on outward sides of the buckle cover for covering the buckle cover.

10. The swimming goggles as claimed in claim 9, wherein the button has a manual portion protruding outward beyond the buckle hole of the buckle cover and cooperating with a protruding portion on the outward cover for facilitating manual operation.

11. The swimming goggles as claimed in claim 1, wherein injecting holes are formed along edges of the eyeglass for plastic passing therethrough when molding.

12. The swimming goggles as claimed in claim 1, wherein a clip is formed on the housing of each strap positioning frame and between the pegs, and is L-shaped for biasing against the eyeglasses inward.

13. The swimming goggles as claimed in claim 9, wherein the connecting portion, the left lens frame and the right lens frame are made of the same soft material and are integrally formed, and the left lens frame and the right lens frame are respectively formed integrally with pads.

14. Swimming goggles comprising a soft left lens frame, a soft right lens frame, a soft connecting portion between the left lens frame and the right lens frame, rigid eyeglasses, rigid strap positioning frames, and fasteners having adjusting buckles with a head strap extending therethrough, the head strap defining stopping grooves therein, the eyeglasses and the strap positioning frames being preliminarily assembled together, the left lens frame and the right lens frame being unitarily formed to connect with each other and enveloping rims of the eyeglasses and parts of the strap positioning frames,
each adjusting buckle including a pressing element pivotable to branches of each strap positioning frame in a leverage fashion, a resilient element for providing return force for the pressing element, and a buckle cover assembling on the branches, the pressing element having an abutting edge on an end thereof for stopping the stopping grooves of the head strap, and a button on another end thereof, the buckle cover defining a buckle hole for corresponding to the button, wherein when the button is pressed, the abutting edge disengages from the head strap to allow adjusting length of the head strap.

15. The swimming goggles as claimed in claim 14, wherein the branches of each strap positioning frame unitarily extend from the housing and are spaced a distance apart from each other, a positioning cover being formed between the branches, a shaft straddling on ends of the branches, an assembling member being formed on an end of the positioning cover and near the housing, a slot being defined adjacent the shaft, the branches respectively define axis holes therein adjacent the shaft for receiving the adjusting buckles.

16. The swimming goggles as claimed in claim 15, wherein the branches of each strap positioning frame respectively define locking holes therein, and the buckle cover of each adjusting buckle forms locking posts for locking with the locking holes.

17. The swimming goggles as claimed in claim 16, wherein the pressing element of each adjusting buckle forms an assembling axis for assembling on the axis holes, and wherein the resilient element of each adjusting buckle includes arcuate levers respectively extending from opposite sides of the assembling axis, support plates respectively extending from the positioning cover, the levers straddling on the support plates, the button remaining a free state in which the button is able to be pressed or return when assembled.

18. The swimming goggles as claimed in claim 14, wherein each adjusting buckle further comprises an outward cover assembled on outward sides of the buckle cover for covering the buckle cover.

19. The swimming goggles as claimed in claim 18, wherein the button of each adjusting buckle has a manual portion protruding outward beyond the buckle hole of the buckle cover and cooperating with a protruding portion on the outward cover for facilitating manual operation.
